(19) United States
(12) Patent Application Publication
(10) Pub. No.: US 2024/0092718 A1
(43) Pub. Date: Mar. 21, 2024

(54) METHODS FOR SYNTHESIZING SUBSTITUTED CARBOXYLIC ACIDS AND PHARMACEUTICALLY ACCEPTABLE SALTS THEREOF

(71) Applicant: HemoShear Therapeutics, Inc., Charlottesville, VA (US)

(72) Inventors: Brad HENKE, Charlottesville, VA (US); Thomas ROPER, Charlottesville, VA (US)

(21) Appl. No.: 18/262,184

(22) PCT Filed: Jan. 20, 2022
(86) PCT No.: PCT/US22/13056

§ 371 Date: Jul. 19, 2023

Related U.S. Application Data
(60) Provisional application No. 63/139,497, filed on Jan. 20, 2021.

(51) Int. Cl. C07C 51/353; C07C 51/48

(52) U.S. Cl. CPC C07C 51/353; C07C 51/48

(57) ABSTRACT

This disclosure provides scalable methods of synthesizing highly pure substituted carboxylic acids. One or more embodiments of the present disclosure relate to methods of synthesizing 2,2-dimethylbutanoic acid.

### CROSS-REFERENCE TO RELATED APPLICATION

The present Application claims priority to U.S. Provisional Patent Application No. 63/139,497, filed Jan. 20, 2021, the entire contents of each of which are incorporated herein by reference and relied upon.

### BACKGROUND OF THE INVENTION

Metabolic disorders occur when there is a mutation in an enzyme that causes a significant loss of function which interrupts the normal flux of metabolites in a metabolic pathway. This results in accumulation of normal intermediary metabolites in abnormally large amounts, and in some cases, the production of abnormal metabolites that are not normally formed.

For example, propionic acidemia (PA) and methylmalonic acidemia (MMA) are inborn errors of metabolism that result from the buildup of metabolites. The incidence rates for PA are 1 in 100,000 individuals in the US, 1 in 50,000 to 100,000 people worldwide, and the incidence can be as high as 1 in 1,000 to 2,000 in specific populations that are genetically at higher risk (e.g., Inuit population of Greenland, some Amish communities, Saudi Arabians, and communities with consanguineous marriage), whereas MMA affects 1 in 48,000 births.

The short chain carboxylic acid, 2,2-dimethylbutanoic acid (HST5040) is a drug candidate for the treatment of PA and MAA. Thus, an improved and scalable synthesis of 2,2-dimethylbutanoic acid to produce a highly pure product is desired.

### SUMMARY OF THE INVENTION

Aspects of the present disclosure provide methods for making 2,2-dimethylbutanoic acid (Compound 10),

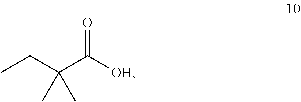

comprising:
(a) allowing isobutyric acid:

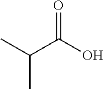

to react with lithium diisopropylamide (LDA) in the presence of an organic solvent at a first temperature ranging from about −10° C. (to about 10° C. to provide a solution comprising an enolate of isobutyric acid; and
(b) allowing the enolate of isobutyric acid to react with CH₃CH₂—X at a second temperature ranging from about 0° C. to about 30° C. to obtain 2,2-dimethylbutanoic acid, wherein X is a halide or leaving group.

In some embodiments, the methods further comprise. (c) adding water to the solution to form an aqueous phase comprising 2,2-dimethylbutanoic acid; and (d) acidifying the aqueous phase to obtain 22-dimethylbutanoic acid.

In some embodiments, the methods further comprise forming a pharmaceutically acceptable salt of 2,2-dimethylbutanoic acid. In some embodiments, the pharmaceutically acceptable salt of 22-dimethylbutanoic acid is a sodium salt.

In some embodiments, the organic solvent comprises tetrahydrofuran (THF), heptane, ethylbenzene, or combinations thereof.

In some embodiments, the methods further comprise adding additional LDA to the solution after step (b). In some embodiments, the methods comprise adding additional LDA to the solution until the solution comprises less than 0.1%, by volume and/or weight, isobutyric acid.

In some embodiments, the methods further comprise the step of: (e) separating the aqueous phase from the solution. In some embodiments, the methods further comprise (f) extracting 2,2-dimethylbutanoic acid from the aqueous phase to an organic phase by adding methyl tert-butyl ether (MTBE) to the aqueous phase.

In some embodiments, the methods further comprise separating unreacted isobutyric acid from 2,2-dimethylbutanoic acid by adding a solution comprising Na₂HPO₄ to the organic phase, wherein the unreacted isobutyric acid is transferred to the solution comprising Na₂HPO₄. In some embodiments, adding the solution comprising Na₂HPO₄ to the organic phase until the organic phase comprises less than 0.1%, by volume and/or weight, isobutyric acid.

In some embodiments, the methods further comprise evaporating MTBE to obtain isolated 2,2-dimethylbutanoic acid.

In some embodiments, the methods further comprise warming the solution in step (a) to a temperature ranging from about 15° C. to about 50° C. In some embodiments, the methods comprise warming the solution in step (a) to a temperature ranging from about 30° C. to about 50° C.

In some embodiments, the methods comprise re-cooling the solution to a temperature ranging from about −10° C. to about 10° C. prior to adding CH₃CH₂—X to the solution.

In some embodiments, the methods further comprise adding water to the solution while maintaining a temperature of the solution at about 30° C. or less.

In some embodiments, the methods comprise acidifying the aqueous phase to a pH ranging from about 1 to about 3. In some embodiments, the methods comprise acidifying the aqueous phase to a pH about 1.

In some embodiments, the methods comprise allowing about 1 molar equivalent of isobutyric acid to react with about 2.5 molar equivalents of LDA.

In some embodiments, a molar excess of (CH₃CH₂—X is used. In some embodiments, the methods comprise reacting about 2 molar equivalents of CH₃CH₂—X with the enolate of isobutyric acid. In some embodiments, after allowing the enolate of isobutyric acid to react with CH₃CH₂—X, the solution comprises less than about 1% of isobutyric acid.

In some embodiments, X is Cl, F, I, or Br. In some embodiments, X is Br.

In some embodiments, the methods allow for a large-scale synthesis of 2,2-dimethylbutanoic acid.

In some embodiments, 2,2-dimethylbutanoic acid is prepared according to the methods of the present disclosure, wherein the 2,2-dimethylbutanoic acid has a purity of about 95% to about 99.9%.

In another aspect, the present disclosure relates to methods of separating isobutyric acid from 2,2-dimethylbutanoic acid, the method comprising.
(a) providing a first solution comprising isobutyric acid and 2,2-dimethylbutanoic acid in an organic solvent; and
(b) adding a second solution comprising Na₂HPO₄ to the first solution, wherein isobutyric acid is transferred from the first solution to the second solution.

In some embodiments, the organic solvent comprises MTBE.

In some embodiments, the second solution is an aqueous solution comprising Na₂HPO₄ at a concentration of about 0.1 M.

In some embodiments, the methods comprise repeating step (b) until all or substantially all of the isobutyric acid is transferred from the first solution to the second solution. In some embodiments, the methods comprise repeating step (b) until the first solution comprises less than 0.1%, by volume and/or weight, isobutyric acid. In some embodiments, the methods comprise repeating step (b) at least one time, at least two times, at least three times, at least four times, or at least live times.

In some embodiments, the methods comprise stirring the first solution and the second solution for about 10 minutes to about 20 minutes.

### DETAILED DESCRIPTION OF THE INVENTION

### Definitions

The term “about” when immediately preceding a numerical value means±up to 20% of the numerical value. For example, “about” a numerical value means±up to 20% of the numerical value, in some embodiments, ±up to 19%, ±up to 18%, ±up to 17%, ±up to 16%, ±up to 15%, ±up to 14%, ±up to 13, ±up to 12%, ±up to 11%, ±up to 10%, ±up to 9%, ±up to 8%, ±up to 7%, ±up to 6%, ±up to 5%, ±up to 4%, ±up to 3%, ±up to 2%, ±up to 1%, ±up to less than 1%, or any other value or range of values therein.

Throughout the present specification, numerical ranges are provided for certain quantities. These ranges comprise all subranges therein. Thus, the range “from 50 to 80” includes all possible ranges therein (e.g. 5.1-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “pharmaceutically acceptable salt” includes both an acid and a base addition salt. Pharmaceutically acceptable salts can be obtained by reacting a compound of the disclosure functioning as a base, with an inorganic or organic acid to form, a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Pharmaceutically acceptable salts can also be obtained by reacting a compound of the disclosure functioning as an acid, with an inorganic or organic base to form a salt, for example, salts of sodium, potassium, lithium, ammonium, calcium, magnesium, iron, zinc, copper, manganese, aluminum, ammonia, isopropylamine, trimethylamine, etc. In some embodiments, the pharmaceutically acceptable salt is a zinc salt. Those skilled in the art will further recognize that pharmaceutically acceptable salts can be prepared by reaction of the compounds of the disclosure with an appropriate inorganic or organic acid or base via any of a number of known methods.

“Alkyl” or “alkyl group” refers to a fully saturated, straight, or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀alkyl includes all moieties described above for C₁-C₅alkyls and C₁-C₆alkyls, but also includes C₇, C₈, C₉ and C₁₀ alkyls. Similarly, a C₁-C₁₂alkyl includes all the foregoing moieties, but also includes C₁₁ and C₁₂ alkyls. Non-limiting examples of C₁-C₁₂alkyl include methyl, ethyl, n-propyl, i-propyl, sec-propyl, n-butyl, i-butyl, sec-butyl, t-butyl, n-pentyl, T-amyl, n-hexyl, n-heptyl, n-octyl, n-nonyl, n-decyl, n-undecyl, and n-dodecyl. Unless stated otherwise specifically in the specification, an alkyl group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic, or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Cycloalkyl” refers to a stable non-aromatic monocyclic or polycyclic fully saturated hydrocarbon consisting solely of carbon and hydrogen atoms, which can include fused or bridged ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Heteroaryl” refers to a 5- to 20-membered ring system comprising hydrogen atoms, one to fourteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazoyl, benzothiazolyl, benzothiadiazolyl, benzo[b][1,4]dioxepanyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, primidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, the “heteroaryl” can be optionally substituted.

The term “substituted” used herein means any of the above groups (e.g., alkyl or aryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diary laminas, N-oxides, imides, and enamines; a silicon atom in groups such as trialkylsilyl groups, dialkylarylsilyl groups, alkyldiarylsilyl groups, and triarylsilyl groups; and other heteroatoms in various other groups, “Substituted” also means any of the above groups in which one or more hydrogen atoms are replaced by a higher-order bond (e.g. a double- or triple-bond) to a heteroatom such as oxygen in oxo, carbonyl, carboxyl, and ester groups: and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

All weight percentages (i.e., “% by weight” and “wt. %” and w/w) referenced herein, unless otherwise indicated, are relative to the total weight of the mixture or composition, as the case may be.

As used herein, an “impurity” is a compound or substance other than the substituted carboxylic acid (e.g., 2,2-dimethylbutyric acid).

As used herein, “isolated” means isolated from a chemical synthesis reaction mixture. In some embodiments, an isolated compound of the disclosure (e.g. 2,2-dimethylbutyric acid) is at least 95% pure and comprises no more than 5% of one or more impurities. By “is at least x % pure” means that a compound of the disclosure includes no more than (100−x) % of one or more impurities. In some embodiments, an isolated compound of the present disclosure is at least 96%, at least 97%, at least 98%, or at least 99% pure, and comprises no more than 4′%, no more than 3%, no more than 2%, or no more than 1% of an impurity, respectively. In some embodiments, an isolated compound of the present disclosure at least 99.5% pure, at least 99.6% pure, at least 99.7% pure, at least 99.8% pure, or at least 99.9% pure, and comprises no more than 0.4%, no more than 0.3%, no more than 0.2%, no more than 0.1%, or no more than 0.01% of an impurity, respectively. In some embodiments, the one or more impurities, if any, are present in the isolated compound of the disclosure as a percent by weight. In some embodiments, the one or more impurities, if any, are present in the isolated compound of the disclosure as a percent by mole. In some embodiments, the one or more impurities, if any, are present in the isolated compound of the disclosure as a, percent by volume.

### Methods of the Disclosure

The present disclosure provides methods for making a compound of Formula B:

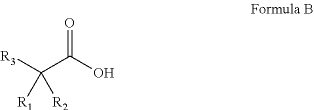

or pharmaceutically acceptable salt thereof,
wherein, R₃ is alkyl, cycloalkyl, aryl, heteroaryl; and
   R₁ and R₂ are each independently H, alkyl, aryl, or heteroaryl,
   or R₃ and R₁ or R₂ together with the carbon atom to which they are bonded form a cycloalkyl;
the method comprising:
   (a) allowing a compound of Formula A:

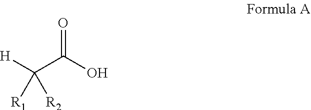

wherein, R₁ and R₂ are each independently H, alkyl, aryl, or heteroaroyl; to react with lithium diisopropylamide (LDA) in the presence of an organic solvent under conditions effective to form a solution comprising an enolate of the compound of Formula A: and
   (b) allowing the enolate to react with R₃—X to obtain a compound of Formula B, wherein X is a halide or a leaving group.

In some embodiments, at least one of R₁ and R₂, is H. In some embodiments, R₁ and R₂ are H. In some embodiments, at least one of R₁ or R₂ is alkyl or aryl. In some embodiments, each of R₁ or R₂ is alkyl or aryl. In some embodiments, each of R₁ and R₂ are alkyl. In some embodiments, the alkyl is a C₁-C₆ alkyl. In some embodiments, the alkyl is a C₁-C₃ alky.

In some embodiments, R₃ is an alkyl, which is optionally substituted. In some embodiments, the substituted alkyl is haloalkyl. In some embodiments, the alkyl is substituted with 1, 2, or 3 halogen. In some embodiments, the halogen is F. In some embodiments, the alkyl is substituted with one or more heteroatoms independently selected from N, O, or S. In some embodiments, substituted alkyl is a C₁-C₆ alkyl-O—C₁-C₃ alkyl, C₁-C₆ alkyl-S—C₁-C₃ alkyl, or C₁-C₆ alkyl-NH—C₁-C₃alkyl. In some embodiments, the alkyl is substituted with —OH. In certain of these embodiments, the —OH can be protected when reacting with the enolate and then deprotected to form a compound of Formula B.

In some embodiments, R₃ is an aryl, which is optionally substituted. In some embodiments, the aryl is substituted with a halogen. In some embodiments, the aryl is substituted with an alkyl.

In some embodiments, R₃ is a cycloalkyl, which is optionally substituted. In some embodiments, R₃ is a cycloalkyl substituted with an alkyl.

In some embodiments, R₁ and R₂ or R₃ together with the carbon atom to which they are bonded from a 3-5-member cycloalkyl ring (e.g., cyclopropyl, cyclobutane, or cycloheptane).

In some embodiments, compounds of Formula B include the compounds in Table 1.

TABLE 1
Compounds of Formula B

| Compound | Structure |
|---|---|
| 1 | 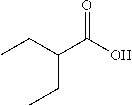 |
| 2 | 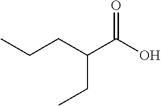 |
| 3 | 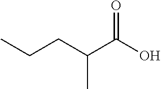 |
| 4 | 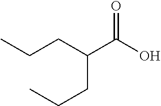 |
| 5 | 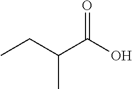 |
| 6 | 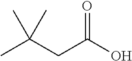 |
| 7 | 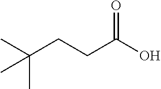 |
| 8 | 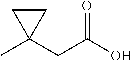 |
| 9 | 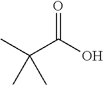 |
| 10 | 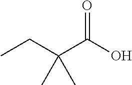 |
| 11 | 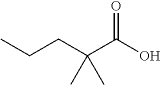 |
| 12 | 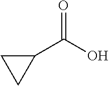 |
| 13 | 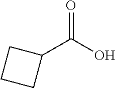 |
| 14 | 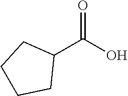 |
| 15 | 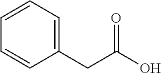 |
| 16 | 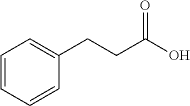 [SMILES: O=C(O)CCc1ccccc1] |
| 17 | 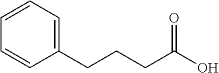 [SMILES: O=C(O)CCCc1ccccc1] |
| 18 | 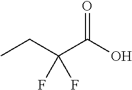 |
| 19 | 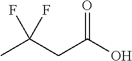 |
| 20 | 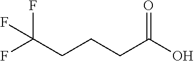 |
| 21 | 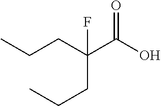 |
| 22 | 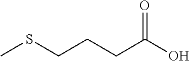 |
| 23 | 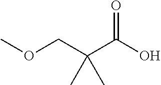 |
| 24 | 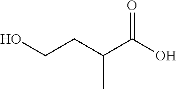 |
| 25 | 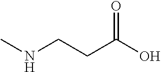 |
| 26 | 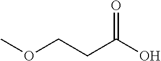 |
| 27 | 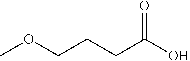 |
| 28 | 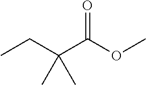 |
| 29 | 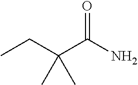 |
| 30 | 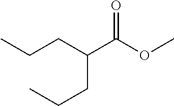 |

In some embodiments, X is a halogen. In certain of these embodiments, the halogen is a fluoride (F, chloride (Cl), bromide (Br), or iodide (I). In some embodiments, the halogen is a bromide (Br). In some embodiments, X is a leaving group. In certain of these embodiments, the leaving group is acetate (Ac)), p-nitrobenzoate (PNBO), or a sulfonate. Non-limiting examples of suitable sulfonates include methanesulfonate (Mesylate: MsO), p-toluenesulfonate (tosylate: TsO), p-bromobenzenesulfonate (Brosylate: BsO), p-nitrobenzenesulfonate (Nosylate: NsO), fluoromethanesulfonate, difluoromethanesulfonate, trifluoromethanesulfonate (Triflate: TfO) and ethanesulfonate. In some embodiments, X is Br.

In some embodiments, the enolate of the compound of Formula A is represented by:

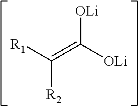

In some embodiments, the compound of Formula B is Compound 10, 2,2-dimethylbutanoic acid (also referred to as 2,2-dimethylbutyric acid, used interchangeably throughout),

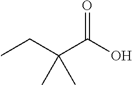

In certain of these embodiments, the compound of Formula A is butyric acid,

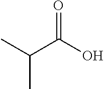

In some embodiments, R₃—X is bromoethane (CH₃CH₂—Br).

In some embodiments, the methods at step (a) comprise allowing a compound of Formula A to react with LDA in the presence of an organic solvent. In some embodiments, the organic solvent comprises tetrahydrofuran (THF), heptane, ethylbenzene, or combinations thereof. In some embodiments, the organic solvent comprises THF, heptane, and ethylbenzene. In certain of these embodiments, a ratio of THF to heptane to ethylbenzene is 1:1:1 by volume, 2:1:1 by volume, 1:2:1 by volume, 1:1:2 by volume, 3:1:1 by volume, 1:3:1 by volume, or 1:1:3 by volume.

In some embodiments, the methods at step (a) comprise allowing a compound of Formula A to react with LDA in the presence of an organic solvent at a first temperature that is greater than −78° C. In some embodiments, the first temperature is greater than −45° C., e.g., about −40° C., about −35° C., about −30° C., about −25° C., about −20° C., about −15° C., about −10° C., about −5° C.-about 0° C., about 5° C., about 10° C., or more, including all values and ranges therebetween. In some embodiments, the first temperature ranges from about −10° C. to about 10° C. to provide a solution comprising an enolate of the compound of Formula A. In certain of these embodiments, the first temperature ranges from about −10° C. to about −5° C., about −10° C. to about 0° C., about 0° C. to about 5° C., about 0° C. to about 10° C., or about 5° C. to about 10° C. In some embodiments, the first temperature is about −10° C., about −5° C., about 0° C., about 5° C., or about 10° C.

In some embodiments, the methods further comprise warming the solution in step (a) to a temperature ranging from about 15° C. to about 50° C. In certain of these embodiments, the methods comprise warming the solution in step (a) to a temperature ranging from about 15° C. to about 30° C., about 15° C. to about 25° C., about 35° C. to about 45° C., about 30° C. to about 35° C., about 35° C. to about 50° C., or about 30° C. to about 40° C. In some embodiments, the methods comprise warming the solution in step (a) to about 15° C., about 20° C., about 25° C., about 30° C., about 35° C., about 40° C., about 45° C., or about 50° C., including all values and ranges therein.

In some embodiments, the methods at step (b) comprise allowing the enolate of the compound of Formula A to react with R₄—X at a second temperature ranging from about 0° C. to about 30° C. to obtain a compound of Formula B. In certain of these embodiments, the second temperature ranges from about 0° C. to about 10° C., about 10° C. to about 20° C., about 10° C. to about 30° C., about 0° C. to about 15° C., or about 10° C. to about 25° C. In some embodiments, the second temperature is about 0° C., about 5° C., about 10° C., about 15° C., about 20° C., about 25′° C., or about 30° C., including all values and ranges therein.

In some embodiments, the methods comprise allowing about 1 molar equivalent of a compound of Formula A to react with about 2 molar equivalents to about 3 molar equivalents of LDA (e.g., about 2, about 2.1, about 2.2, about 2.3, about 2.4, about 2.5, about 2.6, about 2.7, about 2.8, about 29, or about 3 molar equivalents, including all values and ranges therein). In certain of these embodiments, the methods comprise allowing about 1 molar equivalent of a compound of Formula A to react with about 2.5 molar equivalents of LDA. In some embodiments, the methods comprise allowing about 1 molar equivalent of a compound of Formula A to react with about 2 molar equivalents, about 2.1 molar equivalents, about 2.2 molar equivalents, about 2.3 molar equivalents, about 2.4 molar equivalents, about 2.5 molar equivalents, about 2.6 molar equivalents, about 2.7 molar equivalents, about 2.8 molar equivalents, about 2.9 molar equivalents, or about 3 molar equivalents of LDA. In some embodiments, the methods comprise reacting a molar excess of LDA with the compound of Formula A.

In some embodiments, the methods comprise reacting about 2 molar equivalents to about 3 molar equivalents of R₃—X with the enolate of the compound of Formula A (e. g., about 2, about 2.1, about 2.2, about 2.3, about 2.4, about 2.5, about 2.6, about 2.7, about 2.8, about 2.9, or about 3 molar equivalents, including all values and ranges therein). In certain of these embodiments, the methods comprise reacting about 2 molar equivalents of R₁—X with the enolate of the compound of Formula A. In some embodiments, the methods comprise reacting about 2 molar equivalents, about 2.1 molar equivalents, about 2.2 molar equivalents, about 2.3 molar equivalents, about 2.4 molar equivalents, about 2.5 molar equivalents, about 2.6 molar equivalents, about 2.7 molar equivalents, about 2.8 molar equivalents, about 2.9 molar equivalents, or about 3 molar equivalents of R₃—X with the enolate of the compound of Formula A. In some embodiments, the methods comprise reacting a molar excess of R₃—X with the enolate of the compound of Formula A.

In some embodiments, after allowing the enolate to react with R₃—X in step (b) the solution comprises no or substantially no amount of the compound of Formula A. In some embodiments, the solution comprises less than about 1% of the compound of Formula A. In some embodiments, the solution comprises less than about 1%, less than about 0.9%, less than about 0.8%, less than about 0.7%, less than about 0.6%, less than about 0.5%, less than about 0.4%, less than about 0.3%, less than about 0.2%, less than about 0.1%, less than about 0.01%, or less than about 0.001% of the compound of Formula A.

In some embodiments, the methods further comprise adding additional LDA to the solution after step (b). In certain of these embodiments, the methods comprise adding about 0.1 molar equivalents, about 0.2 molar equivalents, about 0.3 molar equivalents, about 0.4 molar equivalents, about 0.5 molar equivalents, about 0.6 molar equivalents, about 0.7 molar equivalents, about 0.8 molar equivalents, about 0.9 molar equivalents, or about 1 molar equivalents of LDA to the solution after step (b).

In some embodiments, the methods further comprise adding additional R₃—X to the solution after the additional LDA is added to the solution after step (b). In certain of these embodiments, the methods comprise adding about 0.1 molar equivalents, about 0.2 molar equivalents, about 0.3 molar equivalents, about 0.4 molar equivalents, about 0.5 molar equivalents, about 0.6 molar equivalents, about 0.7 molar equivalents, about 0.8 molar equivalents, about 0.9 molar equivalents, or about 1 molar equivalents of R₃—X to the solution after the additional LDA is added to the solution after step (b).

In some embodiments, the methods further comprise the steps of: (c) adding water to the solution to form an aqueous phase comprising the compound of Formula B; and (d) acidifying the aqueous phase to obtain the compound of Formula B.

In some embodiments, the methods at step (c) comprise adding water to the solution to form the aqueous phase. In some embodiments, the solution is maintained at a temperature of about 30° C. or less (e.g., about 30° C., about 25° C., about 20° C., about 15° C., about 10° C., about 5° C., or about 0° C., including all values and ranges therein). In certain of these embodiments, the methods comprise maintaining the temperature of the solution at a temperature of about 30° C., about 25° C., about 20° C., about 15° C., about 10° C., about 5° C., about 0° C., or less.

In some embodiments, the methods at step (d) comprise acidifying the aqueous phase to a pH ranging from about 1 to about 3 (e.g., about 1, about 0.5, about 2, about 2.5, or about 5, including all values and ranges therein). In certain of these embodiments, the methods comprise acidifying the aqueous phase to a pH ranging from about 1 to 2 or about 2 to 3. In some embodiments, the methods comprise acidifying the aqueous phase to a pH of about 1, about 1.5, about 2, about 2.5, or about 3. In some embodiments, the methods comprise acidifying the aqueous phase to a pH of about 1.

In some embodiments, the methods at step (d) comprise acidifying the aqueous phase by adding a solution comprising an acid. In some embodiments, the acid is hydrochloric acid (HCl), sulfuric acid (H₂SO₄), or phosphoric acid (H₃PO₄. In some embodiments, a concentration of acid in the solution is about 4 moles/liter of water to about 8 moles/liter of water. In some embodiments, the concentration of the acid is about 4 moles/liter, about 5 moles/liter, about 6 moles/liter, about 7 moles/liter, or about 8 moles/liter of water. In some embodiments, a concentration of acid in the solution is about 6 moles/liter of water.

In some embodiments, the methods further comprise the steps of: (e) separating the aqueous phase from the solution; and (f) extracting the compound of Formula B from the aqueous phase to an organic phase by adding an organic solvent. In certain of these embodiments, the organic solvent comprises methyl/liter-butyl ether (MTBE).

In some embodiments, the methods at step (f) comprise extracting the compound of Formula B from the aqueous phase to the organic phase at least 1, 2, 3, 4, 5, or more times.

In some embodiments, the methods further comprise separating unreacted starting material a compound of Formula A, from a compound of Formula B. In some embodiments, the methods further comprise determining an amount of the compound of Formula A (e.g., starting material) in the organic phase after step (f) and if the organic phase comprises more than about 0.1% of the compound of Formula A, adding water to the organic phase to extract the compound of Formula A from the organic phase to the aqueous phase. In some embodiments, the methods comprise adding water to the organic phase to extract the compound of Formula A at least 1, 2, 3, 4, 5, or more times. In some embodiments, the methods comprise adding water to the organic phase to extract the compound of Formula A until the organic phase comprises less than about 0. % of the compound of Formula A.

In some embodiments, the methods further comprise separating a compound of Formula A from a compound of Formula B comprising adding a solution comprising sodium phosphate to the organic phase, wherein the compound of Formula A is transferred to the solution comprising sodium phosphate. In some embodiments, none or substantially none of the compound of Formula B is transferred to the solution comprising sodium phosphate. In certain of these embodiments, the sodium phosphate comprises NaH₂PO₄, Na₂HPO₄, or combinations thereof. In some embodiments, the sodium phosphate comprises NaH₂PO₄. In some embodiments, the sodium phosphate comprises NaH₂PO₄.

In some embodiments, the methods comprise adding the solution comprising Na₂HPO₄ to the organic phase, mixing the phases together, and separating the two phases, wherein the unreacted starting material is transferred from the organic phase to the aqueous phase. In some embodiments, the method comprises repeating the steps of adding, mixing, and separating 1, 2, 3, 4, 5, 6, 7, 8, or more times. In some embodiments, the methods comprise repeating the adding, mixing, and separating steps until the organic phase comprises less than 0.1% of a compound of Formula A.

In some embodiments, a concentration of Na₂HPO₄ in the solution is about 0.1 moles/liter to about 0.4 moles/liter of water. In some embodiments, the concentration of the sodium phosphate is about 0.1 M. In some embodiments, the concentration of sodium phosphate is about 0.1 moles/liter, about 0.15 moles/liter, about 0.2 moles/liter, about 0.25 moles/liter, about 0.3 moles/liter, about 0.35 moles/liter, or about 0.4 moles/liter of the water.

In some embodiments, the methods further comprise removing (e.g., evaporating) the organic solvent to obtain an isolated compound of Formula B. In some embodiments, the solvent is removed from the compound of Formula B under reduced pressure. In certain of these embodiments, the organic solvent is evaporated from the compound of Formula B using a rotary evaporator.

In some embodiments of the methods of the invention, the methods farther comprise forming a pharmaceutically acceptable salt of a compound of Formula B. In certain of these embodiments, the methods comprise adding a solution of an inorganic or organic base to the compound of Formula B to form a pharmaceutically acceptable salt of a compound of Formula R. In some embodiments, the methods comprise reacting about 1 molar equivalent of the inorganic or organic base with 1 molar equivalent compound of Formula B. In some embodiments, the methods comprise reacting a molar excess of the inorganic or organic base with the compound of Formula B. In some embodiments, the methods comprise forming a sodium, lithium, ammonium, calcium, magnesium, iron, zinc, copper, manganese, aluminum, ammonia, isopropylamine, or trimethylamine salt of a compound of Formula B. In some embodiments, the methods comprise forming a sodium salt of a compound of Formula B. In certain of these embodiments, the methods comprise adding sodium methoxide to the compound of Formula B.

In some embodiments of the methods of the invention, the methods allow for a large-scale synthesis of a compound of Formula B. In certain of these embodiments, the methods can produce at least about 500 g of a compound of Formula B. In some embodiments, the methods can produce at least about 500 g, at least about 550 g, at least about 600 g, at least about 650 g, at least about 700 g, at least about 750 g, at least about 800 g, at least about 850 g, at least about 900 g, at least about 1,000 g, at least about 2.000 g at least about 3,000 g, at least about 4,000 g, at least about 5,000 g, at least about 6,000 g, at least about 7,000 g, at least about 8,000 g, at least about 9,000 g. at least about 10,000 g or more of a compound of Formula B.

In some embodiments, the purity of a compound of Formula Bis at least about 95% (weight %), and a compound of the disclosure comprises no more than about 5% of an impurity (weight %). In some embodiments, the purity of a compound of Formula B is about 95.0% to 100%, and a compound of Formula A (e.g., starting material) comprises 0% to about 5% of an impurity. In some embodiments, the purity of a compound of Formula B is about 98% to 100%, and a compound of Formula A comprises 0% to about 2% of an impurity. In some embodiments, the purity of a compound of Formula B is about 98%, about 98.5%, about 99%, about 99.5%, or 100%, and a compound of Formula A comprises about 2%, about 1.5%, about 1%, about 0.5%, or 0%, respectively, of an impurity, after drying. In some embodiments, the purity of a compound of Formula B is about 99.5%, about 99.9%, or about 99.95%, and a compound of Formula A comprises about 0.5%, about 0.1%, or about 0.05%, respectively, of an impurity, after drying.

In some embodiments, a compound of Formula B comprises less than about 1.5% of an impurity by weight. In some embodiments, a compound of Formula B comprises less than about 1%, less than about 0.5%, less than about 0.4%, less than about 0.3%, less than about 0.2%, less than about 0.1%, less than about 0.01%, less than about 0,001%, less than about 0.0001% of an impurity by weight. In some embodiments, a compound of the disclosure comprises less than 0.1% of an impurity by weight. In some embodiments, the impurity is a compound of Formula A.

In some embodiments, the methods further comprise protecting one or more substituents of a compound Formula A and/or Formula B. A person of ordinary skill in the art would understand the position of attached for the protecting group on a compound of Formula A and/or B as well as an appropriate protecting group to use.

The present disclosure further provides methods for separating a compound of Formula A from a compound of Formula B, the method comprising:
(a) providing a first solution comprising a compound of a compound of Formula B and a compound of Formula A in an organic solvent, and
(b) adding a second solution comprising sodium phosphate to the first solution, wherein the compound of Formula B is transferred from the first solution to the second solution.

In some embodiments of the methods of the invention, the methods comprise repeating step (b) until all or substantially of the compound of Formula A is transferred from the first solution to the second solution. In some embodiments, the methods comprise repeating step (b) 1, 2, 3, 4, 5, or more times. In some embodiments, the methods comprise determining an amount of the compound of Formula A in the first solution after step (b) and if the amount of the compound of Formula A is greater than 0.1%, by weight or volume, of the first solution repeating step (b) until the amount of the compound of Formula A is less than about 0.1%, by weight or volume, of the first solution.

In some embodiments of the methods of the invention, the methods comprise stirring the first solution and the second solution for a time ranging from about 10 minutes to about 20 minutes after step (b).

Pharmaceutical Compositions

The present disclosure also includes pharmaceutical compositions. In some embodiments, a pharmaceutical composition comprises one or more compounds of Formula B or a pharmaceutically acceptable salt thereof. In some embodiments, one or more compounds of Formula B, or a pharmaceutically acceptable salt thereof, in a pharmaceutical composition as described herein, treats a patient suffering from an organic academia, such as PA or MMA.

In some embodiments of the present invention, a pharmaceutical composition comprises a therapeutically effective amount of one or more compounds of Formula B, or a pharmaceutically acceptable salt thereof.

In certain embodiments, a pharmaceutical composition, as described herein, comprises one or more compounds selected from Table 1, or a pharmaceutically acceptable salt thereof.

In some embodiments, a pharmaceutical composition, as described herein, comprising one or more compounds of Formula B, or a pharmaceutically acceptable salt thereof, further comprises one or more additional therapeutically active agents. In one embodiment, one or more additional therapeutically active agents are selected from therapeutics useful for treating PA, MMA, mitochondrial short-chain enoyl-CoA hydratase 1 deficiency (OMIM 616277), 3-hydroxyisobutyryl-CoA hydrolase deficiency (OMIM 250620), 3-hydroxyisobutyrate dehydrogenase deficiency, methylmalonate-semialdehyde dehydrogenase deficiency (OMIM 614105), 2-methyl-3-hydroxybutyryl-CoA dehydrogenase deficiency (OMIM 300438), or 3-methylacetoacetyl CoA thiolase deficiency (OMIM 203750), or combinations thereof.

In further embodiments of the present invention, pharmaceutical compositions comprising one or more compounds of Formula B, or a pharmaceutically acceptable salt thereof, and a pharmaceutically acceptable excipient or adjuvant is provided. The pharmaceutically acceptable excipients and adjuvants are added to the composition or formulation for a variety of purposes. In other embodiments, a pharmaceutical composition comprising one or more compounds of Formula B, or a pharmaceutically acceptable salt thereof further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically active materials such as antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e g., lubricants, preservatives, stabilizers, wetting agents, emulsifiers, salts for influencing osmotic pressure, buffers, colorings, flavorings and/or aromatic substances.

For the purposes of this disclosure, the compounds of the present disclosure can be formulated for administration by a variety of means including orally and parenterally in formulations containing pharmaceutically acceptable carriers, adjuvants, and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions, or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, PA.

In certain embodiments, a pharmaceutical composition of the present disclosure is prepared using known techniques, including, but not limited to mixing, dissolving, granulating, dragee-making, levigating, emulsifying, encapsulating, entrapping, or tableting processes. In various embodiments, the pharmaceutical composition may be selected from the group consisting of a solid, powder, liquid, and a gel. In certain embodiments, the pharmaceutical compositions of the present disclosure are a solid (e.g., a powder, tablet, a capsule, granulates, and/or aggregates).

In some embodiments, the present disclosure provides pharmaceutical compositions comprising a compound of Formula B, or a pharmaceutically acceptable salt thereof, combined with a pharmaceutically acceptable carrier. In some embodiments, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M phosphate buffer or saline (e.g., about 0.8%). Such pharmaceutically acceptable carriers can be aqueous or non-aqueous solutions, suspensions, and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycero, emulsions, or suspensions, including saline and buffered media. Oral carriers can be elixirs, syrups, capsules, tablets, and the like.

Liquid carriers suitable for use in the present application can be used in preparing solutions, suspensions, emulsions, syrups, elixirs, and pressurized compounds. The active ingredient can be dissolved or suspended in a pharmaceutically acceptable liquid carrier such as water, an organic solvent, a mixture of both or pharmaceutically acceptable oils or tats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Liquid carriers suitable for use in the present application include, but are not limited to, water (partially containing additives as above. e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders, or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, tale, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's, and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases, and the like.

Carriers suitable for use in the present application can be mixed as needed with disintegrants, diluents, granulating agents, lubricants, binders, and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient-Common flavoring agents and flavor enhancers, for pharmaceutical products that may be included in the composition and/or combination of the present disclosure include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In certain embodiments, a pharmaceutical composition of the present disclosure is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared using compounds Formula B, or a pharmaceutically acceptable salt thereof, and any other solid excipients where the components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

In some embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiogical saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions, or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions. In various aspects, the amount of the compound of Formula B, or a pharmaceutically acceptable salt thereof, can be administered at about 0.001 mg/kg to about 100 mg/kg body weight (e.g., about 0.01 mg/kg to about 10 mg/kg or about 0, 1 mg/kg to about 5 mg/kg).

Methods of Treatment

In some embodiments, the compounds disclosed herein (i.e., one or more compounds of Formula B or a pharmaceutically acceptable salt thereof) can be used to treat PA, MIA, mitochondrial short-chain enoyl-CoA hydratase 1 deficiency (OMIM 616277), 3-hydroxyisobutyryl-CoA hydrolase deficiency (OMIM 250620), 3-hydroxyisobutyrate dehydrogenase deficiency, methylmalonate-semialdehyde dehydrogenase deficiency (OMIM 614105), 2-methyl-3-hydroxybutyryl-CoA dehydrogenase deficiency (OMIM 300438), or 3-methylacetoacetyl CoA thiolase deficiency (OMIM 203750), or combinations thereof. In certain embodiments, one or more compounds selected from Table 1 can be used to treat PA, MMA, mitochondrial short-chain enoyl-CoA hydratase 1 deficiency (OMIM 616277), 3-hydroxyisobutyryl-CoA hydrolase deficiency (OMIM 250620), 3-hydroxyisobutyrate dehydrogenase deficiency, methylmalonate-semialdehyde dehydrogenase deficiency (OMIM 614105), 2-methyl-3-hydroxybutyryl-CoA dehydrogenase deficiency (OMIM 300438), or 3-methylacetoacetyl CoA thiolase deficiency (OMIM 203750), or combinations thereof.

In some embodiments, the methods comprise administering one or more compounds of Formula B or pharmaceutically acceptable salt thereof. In some embodiments, one or more compounds of Formula B, or a pharmaceutically acceptable salt thereof, in a pharmaceutical composition as described herein, treats a patient suffering from PA or MMA.

In some embodiments of the present invention, a pharmaceutical composition comprises a therapeutically effective amount of one or more compounds of Formula B, or a pharmaceutically acceptable salt thereof.

In certain embodiments, a pharmaceutical composition, as described herein, comprises one or more compounds selected from Table 1, or a pharmaceutically acceptable salt thereof.

In some embodiments, a pharmaceutical composition, as described herein, comprising one or more compounds of Formula B, or a pharmaceutically acceptable salt thereof, further comprises one or more additional therapeutically active agents. In one embodiment, one or more additional therapeutically active agents are selected from therapeutics useful for treating PA, MMA, mitochondrial short-chain enoyl-CoA hydratase 1 deficiency (OMIM 616277), 3-hydroxyisobutyryl-CoA hydrolase deficiency (OMIM 250620), 3-hydroxyisobutyrate dehydrogenase deficiency, methylmalonate-semialdehyde dehydrogenase deficiency (OMIM 614105), 2-methyl-3-hydroxybutyryl-CoA dehydrogenase deficiency (OMIM 300438), or 3-methylacetoacetyl CoA thiolase deficiency (OMI 203750), or combinations thereof.

The methods of the present disclosure can be combined with other therapies used in the treatment of PA or MMA. In some embodiments, such combination therapies entail administering at compound of Formula B in combination with at least one additional therapeutic agent for the treatment of PA and/or MMA. The additional therapeutic agent can be administering subsequently, simultaneously, or sequentially (e.g., before or after) with respect to the IBD and/or MBD inhibitor. Non-limiting examples of additional therapeutic agent which can be combined with the methods disclosed herein include: L-carnitine; ammonia scavengers used to treat acute hyperammonemia, such as N-carbamyl-glutamate, sodium benzoate or sodium phenylbutyrate, antibiotics used to reduce the intestinal flora, such as metronidazole, amoxicillin or cotrimoxazole; vitamin B₁₂ (in B₁₂-responsive MMA patients); biotin; growth hormone therapy: low-protein diets; precursor-free amino acids and/or isoleucine/valine supplements; antioxidant therapies, such as N-acetylcysteine, cystamine or α-tocotrienol quinone; and anaplerotic therapies, such as citrate, glutamine, ornithine α-ketoglutarate or pro-drugs of succinate.

### EXAMPLES

Examples 1-5 provide synthetic details regarding methods of preparing 2,2-dimethylbutyric acid (HST5040) and sodium salt thereof (HST5040A) according to the chemical reaction set forth in Scheme 1,

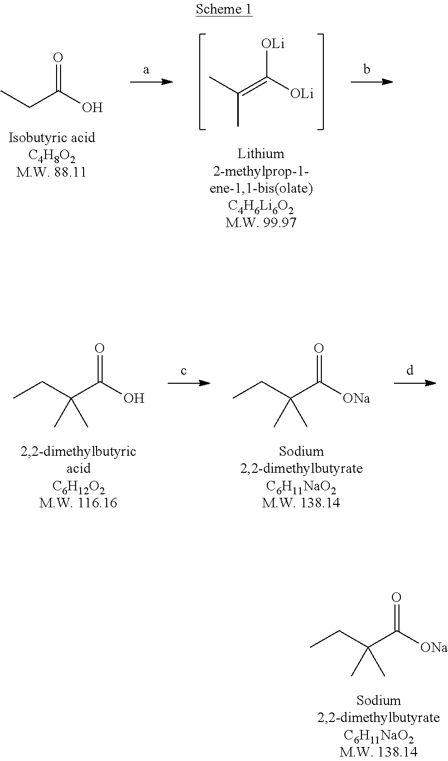

Reagents and Conditions: (a) (1) LDA, THF/heptane/ethylbenzene, 5° C. to 40° C. then recooled to −5° C. to 5° C.; (b) bromoethane, 5° C. to 20° C., then H₂O and HCl: (c) MeONa/MeOH, MTBE then ACN, reflux. (d) ACN/H₂O reflux, then cool to 0° C.

Carboxylic acids have traditionally been alkylated using 2 equivalents of LDA followed by reaction with a corresponding alkyl halide to form 2,2,2-tri substituted carboxylic acids as set forth in Scheme 2.

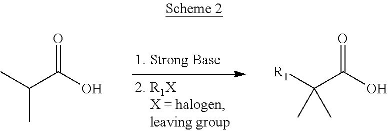

These procedures have had a wide variety of success rates and are often hindered by low yields and require conditions that prevent large scale production of the product. For example, the process for reacting isobutyric acid with propargylic bromide is carried out in benzene-a carcinogenic solvent. In another example, cryogenic techniques are required in order to alkylate isobutyric acid with hexyl iodide. Neither of these procedures are desirable for scale-up due to the use of carcinogenic chemicals and the high cost associated with generating the cryogenic conditions as well as the low yields obtained. While substituted carboxylic acids such as 2,2-dimethyl butyric acid have been previously synthesized by reacting the enolate formed by LDA with an alkyl halides, the enolate is formed at inconveniently low temperatures (−78° C.), which is incompatible with many large-scale reactor configurations as the energy requirement for cooling such a reactor is substantial.

The following examples provide an improved and scalable synthesis for preparing 2,2-dimethylbutyric acid and at a high yield and purity. In particular, the examples demonstrate that upon reacting isobutyric acid with LDA at a temperature of approximately at 0-5° C. (and prior to warming to approximately 40° C.), an enolate of the isobutyric acid is formed, a temperature substantially warmer than that of −78° C. observed in prior methods. The disclosed procedure also uses a solvent combination comprising THF, heptane, and ethyl benzene, the use of which can result in the formation of 2,2-dimethyl butyric acid comprising less than 1% of the starting material, isobutyric acid. Lastly, the synthesis provides an optional step of separating the final product, 2,2-dimethyl butyric acid, from unreacted starting material, isobutyric acid, with the use of a sodium phosphate wash thereby increasing the overall purity and yield of the final product. Taken together, the synthetic procedures described in Examples 1-5 provide an improved and scalable synthesis of 2,2-dimethylbutyric acid and the sodium salt thereof.

### Example 1. Synthesis of 2,2-Dimethylbutyric Acid (HST5040) and Sodium Salt Thereof

The following example provides synthetic details regarding methods of preparing 2,2-dimethylbutyric acid and sodium salt thereof.

Step 1—Synthesis of HST5040: A solution of LDA (24% to 27%) in tetrahydrofuran (THF)/heptane/ethylbenzene (approximately 2.5 equiv; 11.5 wt) is charged to a reactor and cooled to 0-5° C., and then, isobutyric acid (1.0 equiv; 1.0 wt) is added at a rate so as to maintain the reaction temperature below 5° C. The reaction mixture is warmed to between 35° C. and 45° C. before re-cooling the solution to 0-5° C. Bromoethane (approximately 2.0 equiv; 2.5 wt) is charged to the reactor at a rate which maintains the temperature below 5° C., and then, the reaction is warmed to between 15° C. and 25° C. and held until the reaction is complete. The reaction is cooled, and −20 (approximately 10 volumes) is added at a rate so as to maintain the reaction temperature at less than 30° C. The layers are separated, and the product-containing aqueous phase is washed twice with MTBE. The pH of the product-containing aqueous layer is adjusted to approximately pH=1 by the addition of 6 N aqueous HCl while maintaining the temperature at less than 30° C. The product-containing acidic aqueous phase is extracted three times with MIE to provide a solution of HST5040 in the organic phase. The product-containing organic phase is washed twice with H₂O, and then, a sample is removed for determination of isobutyric acid content. If the solution contains more than 0.1% isobutyric acid, additional H₂O washes are conducted. Once the level of isobutyric acid is <0.1%, MTBE is added, and the solvent is evaporated to a minimum. An additional charge of MTBE is added, and the additional solvent is evaporated to afford a solution of HST5040 in MTBE (about 50% wt/wt). The product is carried on to step 2 (expected yield 75-95% theory) without further isolation.

The synthetic processes set forth in step 1 was significant in that it afforded a higher conversion rate and required less severe conditions as compared to alternative processes to affect the alkylation of isobutyric acid to 2,2-dimethylbutyric acid. For example, as shown in Table 2, minor changes to the synthetic conditions did not yield as desirable results as the combination of conditions set forth in step 1. In particular, the electrophilic alkylating agent, ethyl bromide yielded a higher conversion of isobutyric acid to 2,2-dimethylbutyric acid as compared to both ethyl tosylate and ethyl chloride. In addition, use of the lithium amide base, LiHMDS, resulted in a low conversion of isobutyric acid to 2,2-dimethylbutyric acid, which contrasts with the high conversion observed in the present example with the use of LDA. Lastly, lower alkylating temperatures (e.g., −78° C.) yielded a lower conversion of isobutyric acid to 2,2-dimethylbutyric acid as compared to higher conversion temperatures (e.g., −45° C.).

TABLE 2
Comparative Conditions to the Reaction of Example 1

| Condition | Alternative Conditions | Result |
|---|---|---|
| Electrophilic | Ethyl tosylate | 2% isobutyric acid remaining |
| alkylating agent | Ethyl chloride | 60% of isobutyric acid remaining |
| Lithium Amide Base | Lithium bis(trimethylsilyl)amide | Low conversion of isobutyric acid |
| | (LiHMDS) | to 2,2-dimethylbutyric acid |
| Alkylation | −45° C. | 3% isobutyric acid remaining |
| temperature | −78° C. | 24% of isobutyric acid remaining |

Step 2—Synthesis of HST5040A: To a solution of HST5040 in MTBE (1 weight, about 50% wt/wt in solution) is added approximately 2 volumes of MTBE. The solution is cooled to 0-5° C., and a solution of sodium methoxide in methanol (25% wt/wt, 0.95 equiv, 1.0.77 w eights) is added while maintaining the temperature below 20° C. The sodium salt of 2,2-dimethylbutyric acid (HST5040K) is then isolated from the solution.

### Example 2. Synthesis of 2,2-Dimethylbutyric Acid (HST5040) and Sodium Salt Thereof (HST5040A)—Sodium Phosphate Extraction

The following example provides synthetic details regarding methods of preparing 2,2-dimethylbutyric acid and sodium salt thereof similar to that set forth in Example 1 with an additional and optional step of removing unwanted starting material via a sodium phosphate extraction.

Step 1—Synthesis of HST5040: A solution of LDA (24%-27%) in THF/heptane/ethylbenzene (approximately 2.75 eq; 12.5 wt) is charged to a reactor and cooled and then isobutyric acid (10 eq; 1.0 wt) is added at a rate so as to maintain the reaction temperature below 5° C. The reaction mixture is warmed to about 35-45° C. before re-cooling the solution. Bromoethane (approximately 2.2 eq; 2.75 wt) is charged to the reactor at a rate which maintains the temperature below 5° C. and then the reaction is warmed to about 1.5-25° C. and held until the reaction is complete. The reaction is cooled, and water (approximately 10 volumes) are added, maintaining the reaction temperature <30° C. The layers are separated, and the product-containing aqueous phase is washed twice with MTBE. The pH of the product containing aqueous layer is adjusted to about pH 1 by the addition of 6 N aqueous HCO while maintaining the temperature 30° C. The product-containing acidic aqueous phase is extracted three times with MTBE to provide a solution of HST5040 in the organic phase. The product-containing organic phase is washed twice with water and then a sample is removed for determination of isobutyric acid content.

Step 2—Sodium Phosphate Extraction: if the solution contains more than 0.1% isobutyric acid, disodium phosphate (Na₂HPO₄) aqueous solution (0.1 M) washes are conducted. Washes with Na₂HPO₄ solutions were determined to remove substantially all the starting material, isobutyric acid, from the organic phase comprising 2,2-dimethyl-butyric acid. For example, Table 3 shows the relative reduction of isobutyric acid and increase in the level of 2,2-dimethylbutyric acid upon the Na₂HPO₄ wash with two test samples (“test sample” and “test sample 2”).

TABLE 3
Analysis of the effect of extraction with
Na₂HPO₄ on the amounts of isobutyric acid and
2,2-dimethylbutyric acid on test samples in the organic layer

| | | Isobutyric | 2,2- |
|---|---|---|---|
| Sample | | acid | dimethylbutyric |
| name | Description | (area %) | acid (areas %) |
| Test | Initial level of isobutyric acid | 0.33 | 99.52 |
| Sample 1 | After first 0.1M Na₂HPO₄ wash | 0.16 | 99.69 |
| | After second 0.1M Na₂HPO₄ | 0.07 | 99.76 |
| | wash | | |
| Test | Initial level of isobutyric acid | 0.73 | 98.89 |
| Sample 2 | After first 0.1M Na₂HPO₄ wash | 0.41 | 99.19 |
| | After second 0.1M Na₂HPO₄ | 0.22 | 99.33 |
| | wash | | |
| | After third 0.1M Na₂HPO₄ wash | 0.09 | 99.44 |

This process has advantages over extractions with other aqueous washes, where both 2,2-dimethylbutyric acid and the isobutyric acid are extracted into the water layer thereby preventing the separation of the two species. For example, Table 4 enumerates the results from washing the organic layer comprising both 2,2-dimethylbutyric and isobutyric acid with aqueous washes comprising only water, sodium bicarbonate (NaHCO₃), ammonium chloride (NH₄Cl), HCl. and brine (sodium chloride (NaCl)). As shown in Table 4, significant amounts of 2,2-dimethylbutyric acid and isobutyric acid were extracted from the organic layer into the aqueous layer, preventing optimal separation of the two species. Specifically, water, HCl, NH₄Cl, and brine provided no selectivity between 2,2-dimethylbutyric acid and the isobutyric acid and while NaHCO₃ provided some amount of selectivity, a significant amount of 2,2-dimethylbutyric acid was lost to the aqueous layer. In contrast, the use Na₂HPO₄ selectively separated isobutyric acid from the similarly structured product, 2,2-dimethyl-butyric acid without significant loss of the product to the water layer (Table 3 and Table 4) thereby increasing the overall purity and yield of the final product. This process also offers advantages over existing separation technologies that rely on cumbersome techniques requiring the use of supercritical CO₂, organic solvents, and electrodialysis to affect the separation of the two carboxylic acid species

TABLE 4
Analysis of the effect of extraction with aqueous washes
on the amounts of isobutyric acid and 2,2-dimethylbutyric
acid extracted into the aqueous layer

| MTBE | | Isobutyric | 2,2- | 2,2- |
|---|---|---|---|---|
| layer | Aqueous | acid | dimethylbutyric | dimethylbutyric |
| sample # | Solution | (arca %) | acid (area %) | acid (area %)^{b} |
| HUA- | Starting | 44.22 | 55.78 | 1885 |
| AC-32-1 | mixture | | | |
| HUA- | Water | 41.72 | 58.28 | 1748 |
| AC-32-2 | | | | |
| HUA- | 5% NaHCO₃ | 16.01 | 83.99 | 1233 |
| AC-32-3 | (pH 8) | | | |
| HUA- | 0.1M Na₂HPO₄ | 36.94 | 63.06 | 1887 |
| AC-32-4 | (pH 9) | | | |
| HUA- | Saturated | 43.61 | 56.84 | 1631 |
| AC-32-5 | NH₄Cl | | | |
| | (pH 4.5) | | | |
| HUA- | 2N HCl | 42.16 | 57.84 | 1970 |
| AC-32-6 | (pH <0) | | | |
| HUA- | Brine | 43.98 | 56.02 | 1923 |
| AC-32-7 | | | | |

Accordingly, a 20 mL HST5040 solution in MTBE from the reaction above was charged 0.1M Na₂HPO₄ aqueous solution (160 mL). The mixture was stirred for 10-20 minutes, settled for 5-10 minutes, and the aqueous layers removed. This process was then repeated 5 tines whereby the isobutyric acid level in MTBE layer was reduced from 3.46% before wash to 0.07% after the fifth wash (Table 5).

TABLE 5
Analysis of the effect of extraction with Na₂HPO₄ on
the amounts of isobutyric acid and 2,2-dimethylbutyric
acid on the sample of Example 2 in the organic layer

| | Isobutyric | |
|---|---|---|
| | acid | 2,2-dimethylbutyric |
| Description | (area %) | acid (area %) |
| MTBE layer before wash | 3.46 | 96.31 |
| MTBE layer after 1st Na₂HPO₄ wash | 2.12 | 97.59 |
| MTBE layer after 2nd Na₂HPO₄ wash | 1.27 | 98.47 |
| MTBE layer after 3rd Na₂HPO₄ wash | 0.52 | 99.25 |
| MTBE layer after 4th Na₂HPO₄wash | 0.21 | 99.59 |
| MTBE layer after 5th Na₂HPO₄wash | 0.07 | 99.96 |

Once the level of isobutyric acid is <01%, MTBE is added, and the solvent is evaporated to a minimum. An additional charge of MTBE is added, and additional solvent is evaporated to afford a solution of HST5040 in MTBE (about 30-50% wt/wt). The product is then carried, on to step 3 (expected yield 60-95%) without further isolation.

Step 3-Synthesis of HST5040A: To a solution of HST5040 in MTBE (1 weight, about 30-50% wt/wt in solution) is added approximately 2 volumes of MTBE. The solution is cooled and a solution of sodium methoxide in methanol (25% wt/wt 0.95 eq, 1.77 weights) is added while maintaining the temperature below 20° C. The sodium salt of 2,2-dimethylbutyric acid (HST5040A) is then isolated from the solution.

### Example 3. Synthesis of 2,2-dimethylbutyric Acid (HST15040) and Sodium Salt Thereof (HST5040A)—Additional LDA Charge

The following example provides synthetic details regarding methods of preparing 2,2-dimethylbutyric acid and sodium salt thereof similar to that set forth in Examples 1 and 2 with an additional and optional step of adding LDA after the addition of bromoethane to ensure completion of the reaction.

Step 1—Synthesis of HST5040: LDA (2.75 equiv, 780 mL) was added to a 1 L jacketed reactor charged with nitrogen. The temperature was adjusted to 0±5° C. and isobutyric acid (1 equiv, 50 g) was added over 1 hour using a syringe pump while maintaining the temperature. A 0.08 vol (4 mL) rinse with anhydrous THE followed. The mixture was heated to 40° C. over 1 hour and then, stirred for an additional hour. The temperature was adjusted to 0±5° C. and bromoethane (2.20 equiv, 136 g) was added subsurface over 1.5 hours using a syringe pump. A 0.08 vol (8 m L) rinse with anhydrous THF followed. The temperature was then adjusted to 20±5° C. After stirring for 18 h, the reaction appeared off-white slurry and contained 0.40 area % isobutyric acid. An additional 10 mol % solution of LDA (78 m-L) was added to the reaction at room temperature and the reaction was stirred for 10 minutes followed by a 10 mol % solution of bromoethane (13.7 g). The reaction was left to stir for 56 hours. The reaction was then acidified, and the product-containing aqueous phase was washed with 4 volumes of MTBE.

Step 2—Sodium Phosphate Extraction: The MTBE organic layer was then washed with two, 8 volumes of 0.1 M Na₂HPO₄ in order to reach an amount of isobutyric acid <0.1%. A water wash followed, and the MTBE layer was concentrated to 4 volumes. An additional distillation was carried out under vacuum to bring down water content within the specification (Karl Fischer<2%). Calculated yield of was 50.7 g (77% yield, 0.07% isobutyric acid).

Step 3—Synthesis of HST5040: The reaction mixture was diluted to 6 volumes using MTBE and cooled to 0° C. Sodium methoxide in methanol (25 wt %) was added slowly by addition funnel over 1 h maintaining temperature 120° C. The reaction mixture was concentrated by distillation to 3 volumes. The sodium salt of 2,2-dimethylbutyric acid (HST5040A) was then isolated from the solution.

### Example 4. Large Scale Synthesis of 2,2-dimethylbutyric Acid (HST5040) and Sodium Salt Thereof (HST5040A)

The following example provides synthetic details regarding methods of preparing 2,2-dimethylbutyric acid and sodium salt thereof similar to that set forth in Example 1 on a large scale.

Step 1—Synthesis of HST5040: To a reactor at 0° C. was added 8000 L. (16000 mmol, 2.6 eq) of 2M lithium diisopropyl amide in THF/heptane/ethylbenzene. The temperature was reduced and isobutyric acid (580 mL, 540 g, 6128 mmol, 1 equivalent) was added over the course of 2 hours and then the temperature was increased to 40¹C for 1-2 hours. The temperature was reduced to 0° C. and bromoethane (950 mL, 1.4 kg, 12847 mmol, 2.1 eq) was added over the course of approximately 4 hours. The reaction was warmed to ambient temperature and stirred for 1-2 days. Water was added to the reaction mixture and the layers were separated. The lower, product containing, aqueous layer was extracted with MTBE at least once. The product containing aqueous layer was acidified with cooling using 6N HC. The acidified aqueous later was extracted several times with MTBE. The combined MTBE extracts were wasted with water and the solvent was evaporated. Additional MTBE was added, and the solvent was evaporated again.

Step 2—Synthesis of HST5040A: AMIE was added, and the mixture was cooled to 0° C. before the addition of sodium methoxide (25 wt %, 1330 mL, 5817 mmol, 0.95 eq) was added and the solvent was evaporated. Alternatively, a lower amount of sodium methoxide can be added to improve the overall quality of the final product. Acetonitrile was added and then the solvent was evaporated. Additional acetonitrile was added, and the solvent was again evaporated. Additional charges of acetonitrile may be needed in order to remove the methanol and MTBE. The sodium salt of 2,2-dimethylbutyric acid (HST5040A) was then isolated from the solution.

### Example 5. Large Scale Synthesis of 2,2-dimethylbutyric Acid (HST5040) and Sodium Salt Thereof (HST5040A)—Sodium Phosphate Extraction

The following example provides synthetic details regarding methods of preparing 2,2-dimethylbutyric acid and sodium salt thereof similar to that set forth in Examples 1-4 on a large scale with an additional and optional step of removing unwanted starting, material via a sodium phosphate extraction.

Step 1 Synthesis of HST5040: A solution comprising 2 M LDA in THF/heptane ethylbenzene was added to 200-L jacketed Hastelloy reactor under an inert atmosphere (62.5 kg 78 L, 156 mol, 2.75 equiv-, RM-13201) at 0+5° C. isobutyric acid (5000 g, 56.7 mol, RM-13167) was added over 3 h and 39 min with an anhydrous THF (400 mL, RM-13094) rinse while maintaining the temperature at ≤5° C. The mixture was heated to 40±5° C. over 1 h and 5 min and stirred for 1 h and 8 min; an orange precipitate was observed to form during stirring. The mixture was then cooled to 0±5° C. over 1 h and 54 min.

Bromoethane (9.3 L, 125 mol, 2.2 equiv-; RM-13090, RM-13175) was then added over 6 h and 43 min using an anhdrous THF (400 mL, RM-13094) rinse while maintaining a temperature at 0-5° C. The batch was heated to 20a 5° C. over 24 min and stirred for 15 h and 44 mint. Analysis of the mixture taken at 12 h and 26 min indicated that the reaction was not complete with 6.8% isobutyric acid observed (specification: Isobutyric acid 50.1% by conversion to HST5040). Additional 2 M LDA (7.8 L, 15.6 mol, 0.27.5 equiv., RM-1-32011) was added using a THF (500 mL) rinse. The mixture was heated to 40±5° C. over 35 min, stirred for 5 h, and cooled 3.5 to 20±5° C. over 45 min. Analysis of the batch after stirring at 20±5° C. for 9 h and 17 min indicated that the reaction was not complete with 2.0% isobutyric acid observed.

The batch was cooled to 5 5° C. over 55 min. Purified water (50 L) was added over 13 min while maintaining a temperature at <30° C. The phases were separated and removed from the reactor. The rich aqueous phases were returned to the reactor and washed twice with MTBE (50 L. and 25 L) with stirring each for 15 min. A 6 M HCl solution (11.5 L, 69 mol, 1.22 equiv, made from concentrated HCl (10 L) and purified water (10 L)) was added portion wise over about 28 min until a pH S 2 was observed. The layers were separated and removed and the aqueous was extracted with MTBE (20 L). The batch was sampled and determined to contain 2.3% isobutyric acid by conversion to 2,2-dimethyl-butanoate.

Step 2—Sodium Phophate Extraction: The organic layer was washed 5 times with 0.1 M Na₂HPO₄ Solution (5×40 L, made from Na₂HPO₄ and purified water) and with purified water (40 L). Analysis of the batch after the 5th sodium phosphate wash indicated that the levels of isobaric acid were 0.05%.

Step 3—Synthesis of HST5040A: The batch was concentrated under vacuum to a volume of 20 L over about 1 h. MTBE (25 L) was added, and the batch was concentrated under vacuum to a volume of 9.5 L over 24 min; MTBE (15.5 L) was added to the reactor to reach the target volume with 25 L measured in the reactor. The batch was removed from the reactor via a 10 μm in-line filter using MTBE (5 L) and weighed (23.50 kg). The mixture was analyzed for wt % assay and determined to contain 18.8% HST5040 by weight in solution (corresponding to a total of about 4418 g (67% yield) of HST5040). The batch was returned to the reactor using MTBE (5 L) and this mixture was concentrated under vacuum to a volume of 29 L over about 30 min. The mixture was cooled to 01.5° C. over 56 min and 25% NaOMe (7785 g, 36.0 mol, 0947 equiv) was added over 30 min at a temperature <20° C. using a MeOH (400 mL, RM-12361) rinse. The sodium salt of 2,2-dimethylbutyric acid (HST5040A) was then isolated from the solution.

1. A method of synthesizing 2,2-dimethylbutanoic acid (Compound 10), 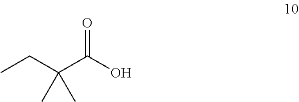
the method comprising:
(a) allowing isobutyric acid: 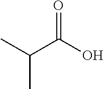
to react with lithium diisopropylamide (LDA) in the presence of an organic solvent at a first temperature ranging from about −10° C. to about 10° C. to provide a solution comprising an enolate of isobutyric acid; and
(b) allowing the enolate of isobutyric acid to react with CH₃CH₂—X at a second temperature ranging from about 0° C. to about 30° C. to obtain 2,2-dimethylbutanoic acid, wherein X is a halide or leaving group.

2. The method of claim 1, further comprising
(c) adding water to the solution to form an aqueous phase comprising 2,2-dimethylbutanoic acid; and
(d) acidifying the aqueous phase to obtain 2,2-dimethylbutanoic acid.

3. The method of claim 1, further comprising forming a pharmaceutically acceptable salt of 2,2-dimethylbutanoic acid.

4. The method of claim 3, wherein the pharmaceutically acceptable salt of 2,2-dimethylbutanoic acid is a sodium salt.

5. The method of claim 1, wherein the organic solvent comprises tetrahydrofuran (THF), heptane, ethylbenzene, or combinations thereof.

6. The method of claim 1, further comprising adding additional LDA to the solution after step (b).

7. The method of claim 6, further comprising adding additional LDA to the solution until the solution comprises less than 0.1%, by volume and/or weight, isobutyric acid.

8. The method of claim 1, further comprising the step of:
(e) separating the aqueous phase from the solution.

9. The method of claim 8, further comprising the step of:
(f) extracting 2,2-dimethylbutanoic acid from the aqueous phase to an organic phase by adding methyl tert-butyl ether (MTBE) to the aqueous phase.

10. The method of claim 9, further comprising separating unreacted isobutyric acid from 2,2-dimethylbutanoic acid by adding a solution comprising Na₂HPO₄ to the organic phase, wherein the unreacted isobutyric acid is transferred to the solution comprising Na₂HPO₄.

11. The method of claim 10, further comprising adding the solution comprising Na₂HPO₄ to the organic phase until the organic phase comprises less than 0.1%, by volume and/or weight, isobutyric acid.

12. The method of claim 9, further comprising evaporating MTBE to obtain isolated 2,2-dimethylbutanoic acid.

13. The method of claim 1, further comprising warming the solution in step (a) to a temperature ranging from about 15° C. to about 50° C.

14. The method of claim 13, further comprising warming the solution in step (a) to a temperature ranging from about 30° C. to about 50° C.

15. The method of claim 14, further comprising re-cooling the solution to a temperature ranging from about −10° C. to about 10° C. prior to adding CH₃CH₂—X to the solution.

16. The method of claim 1, comprising adding water to the solution while maintaining a temperature of the solution at about 30° C. or less.

17. The method of claim 2, comprising acidifying the aqueous phase to a pH ranging from about 1 to about 3.

18. The method of claim 2, comprising acidifying the aqueous phase to a pH about 1.

19. The method of claim 1, wherein a molar excess of LDA is used in step (a).

20. The method of claim 1, comprising allowing about 1 molar equivalent of isobutyric acid to react with about 2.5 molar equivalents of LDA.

21. The method of claim 1, wherein a molar excess of CH₃CH₂—X is used.

22. The method of claim 1, comprising reacting about 2 molar equivalents of CH₃CH₂—X with the enolate of isobutyric acid.

23. The method of claim 1, wherein after allowing the enolate of isobutyric acid to react with CH₃CH₂—X, the solution comprises less than about 1% of isobutyric acid.

24. The method of claim 1, wherein X is Cl, F, I, or Br.

25. The method of claim 24, wherein X is Br.

26. The method of claim 1, wherein the method allows for a large-scale synthesis of 2,2-dimethylbutanoic acid.

27. 2,2-dimethylbutanoic acid prepared according to the process of any claim 1, wherein the 2,2-dimethylbutanoic acid has a purity of about 95% to about 99.9%.

28. A method of separating isobutyric acid from 2,2-dimethylbutanoic acid, the method comprising:
(a) providing a first solution comprising isobutyric acid and 2,2-dimethylbutanoic acid in an organic solvent; and
(b) adding a second solution comprising disodium phosphate (Na₂HPO₄) to the first solution, wherein isobutyric acid is transferred from the first solution to the second solution.

29. The method of claim 28, wherein the organic solvent comprises methyl tert-butyl ether (MTBE).

30. The method of claim 28, wherein the second solution is an aqueous solution comprising Na₂HPO₄ at a concentration of about 0.1 M.

31. The method of claim 28, further comprising repeating step (b) until all or substantially all of the isobutyric acid is transferred from the first solution to the second solution.

32. The method of claim 28, further comprising repeating step (b) until the first solution comprises less than 0.1%, by volume and/or or weight, isobutyric acid.

33. The method of claim 28, further comprising repeating step (b) at least one time, at least two times, at least three times, at least four times, or at least five times.

34. The method of claim 28, further comprising stirring the first solution and the second solution for about 10 minutes to about 20 minutes.
